(19) United States
(12) Patent Application Publication
(10) Pub. No.: US 2020/0148997 A1
(43) Pub. Date: May 14, 2020

(54) METHOD OF PRODUCING HIGH FUCOXANTHIN-CONTAINING ALGA

(71) Applicants: South Product Ltd., Uruma-shi (JP); JXTG Nippon Oil & Energy Corporation, Chiyoda-ku (JP)

(72) Inventors: Takayuki KANESHIRO, Okinawa (JP); Masahiko IHA, Okinawa (JP); Toshiyuki TAKAHASHI, Tokyo (JP); Takashi ISHIBASHI, Tokyo (JP)

(73) Assignees: South Product Ltd., Uruma-shi (JP); JXTG Nippon Oil & Energy Corporation, Chiyoda-ku (JP)

(21) Appl. No.: 16/677,815

(22) Filed: Nov. 8, 2019

(30) Foreign Application Priority Data
Nov. 9, 2018 (JP) 2018-211560

(51) Int. Cl. C12N 1/12; C12M 1/12; C12M 1/00

(52) U.S. Cl. CPC C12N 1/12; C12M 31/10; C12M 37/00

(57) ABSTRACT

Provided is a method of producing a high fucoxanthin-containing alga, the method including culturing an alga containing fucoxanthin by irradiating the alga with light from a light source capable of emitting at least light corresponding to green. This method is capable of increasing fucoxanthin production.

### BACKGROUND OF THE INVENTION

### Field of the Invention

The present invention relates to production of a high fucoxanthin-containing alga in which the fucoxanthin content is enhanced.

### Description of the Related Art

Fucoxanthin is one of carotenoids which is represented by the following formula. Fucoxanthin is reported to have a strong antioxidant, anti-obesity, antidiabetic, and other actions (Non-patent Document 1) and is expected as a functional carotenoid that follows astaxanthin.

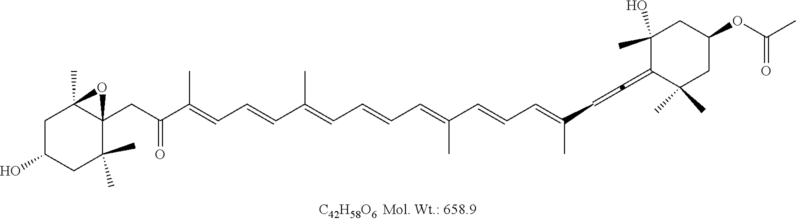

Although fucoxanthin is contained in algae, for example, microalgae, such as diatoms, and seaweeds, such as brown algae, the amount is small. This has been a reason for the unexpanded use of fucoxanthin.

### RELATED ART DOCUMENT

### Patent Document

Patent Document 1: Japanese Patent Application No. 2009-009456

Patent Document 2: Japanese Patent Application No. 2009-211721

### Non-Patent Document

Non-patent Document 1: MAEDA et al: International Journal of Molecular Medicine, 18, 147-152, 2006

Non-patent Document 2: IHA, Masahiko “Kaisou no Baiyou niyoru Karotenoido no Seisan (Production of carotenoids by culturing seaweeds)”, Journal of Bioscience and Bioengineering, 93, 7, 2015

### SUMMARY OF THE INVENTION

An object of the present invention is to provide a technique of increasing fucoxanthin production.

As a result of intensive studies for achieving the object, the present inventors have found that fucoxanthin contained in an alga containing fucoxanthin can be enriched by culturing the alga by irradiating the alga with specific light, thus completing the present invention.

That is, the present invention is directed to a method of producing a high fucoxanthin-containing alga, the method including culturing an alga containing fucoxanthin by irradiating the alga with light from alight source capable of emitting at least light corresponding to green.

The present invention is also directed to a high fucoxanthin-containing alga produced by the production method.

The present invention is also directed to a method of preventing contamination with an alga other than an alga containing fucoxanthin, the method including culturing an alga containing fucoxanthin by irradiating the alga with light from alight source capable of emitting at least light corresponding to green.

The present invention is also directed to an apparatus for producing a high fucoxanthin-containing alga, the apparatus including a culture vessel and a light source capable of emitting at least light corresponding to green.

The method of producing a high fucoxanthin-containing alga of the present invention makes it possible to produce a high fucoxanthin-containing alga in which fucoxanthin contained in an alga is enriched with a simple apparatus in an efficient manner.

The high fucoxanthin-containing alga is highly helpful for expanding the use of fucoxanthin.

### BRIEF DESCRIPTION OF THE DRAWINGS

FIG. 1 illustrates a spectrum of a green LED that is preferred in the production method of the invention.

FIG. 2 illustrates a spectrum of a white LED that is preferred in the production method of the invention.

FIG. 3 is a schematic view of a culture apparatus used in Example 1.

FIG. 4 illustrates spectra of LEDs used in Example 1.

FIG. 5 illustrates a variation in mass of an alga body in Example

FIG. 6 illustrates a variation in fucoxanthin content per unit mass in Example 1.

FIG. 7 illustrates a variation in fucoxanthin productivity per culture volume in Example 1.

FIG. 8 illustrate a relationship between light source wavelength and chromogenesis in Example 1.

FIG. 9 illustrates a relationship between light source wavelength and chromogenesis in Example 1.

FIG. 10 illustrates a variation in alga body mass, a variation in fucoxanthin content per unit mass, and a variation in fucoxanthin productivity per culture volume by light intensity in Example 3.

FIG. 11 illustrates a relationship between light intensity and chromogenesis in Example 3.

FIG. 12 illustrates a relationship between light intensity and chromogenesis in Example 3.

### DETAILED DESCRIPTION OF THE INVENTION

The method of producing a high fucoxanthin-containing alga of the present invention (hereinafter referred to as “the production method of the invention”) is a method including culturing an alga containing fucoxanthin by irradiating the alga with light from a light source capable of emitting at least light corresponding to green.

The alga containing fucoxanthin used in the production method of the invention is not particularly limited. Examples of such algae include: diatoms, such as genus phaeodactylum and genus thalassiosira; unicellular haptocytes and raphidophytes belonging to genus isochrysis; microalgae, such as dinoflagellates; and seaweeds, for example, brown algae, such as Okinawa mozuku (Cladosiphon okamuranus), mozuku or ito-mozuku (Nemacystus decipiens), habanori (Petalonia binghamiae), wakame (Undaria pinnatifida), kombu (Laminariaceae), and hondawara (Sargassum fulvellum). Among the algae, a brown alga or a diatom is preferred, Okinawa mozuku, mozuku, or Habanori is more preferred, and Okinawa mozuku or mozuku is particularly preferred. The form of an alga used in culture is not particularly limited, and a discoidal form and a filamentous form are preferred.

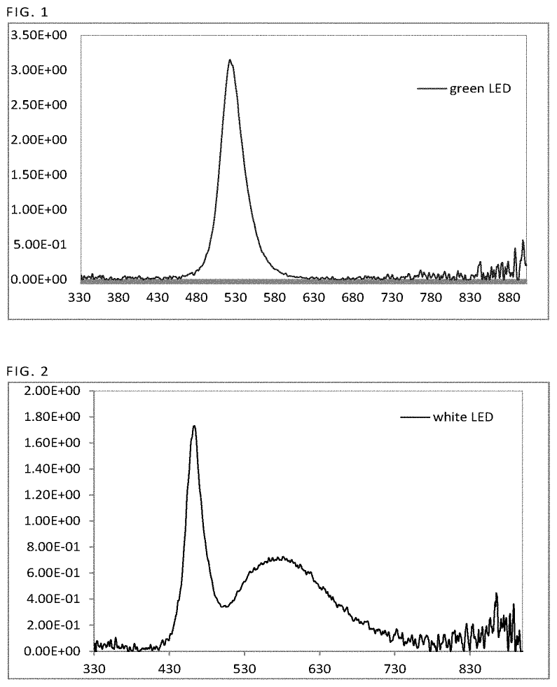

The light source capable of emitting at least light corresponding to green for use in the production method of the invention is not particularly limited, and preferable examples of such light sources include a green LED which mainly emits light of wavelengths corresponding to green (500 to 560 nm) and a white LED which emits light that contains light corresponding to green and that looks white in combination with light corresponding to other colors. An example of a spectrum of a green LED is shown in FIG. 1 and an example of a spectrum of a white LED is shown in FIG. 2. Such light sources are available from various LED manufacturers. In the production method of the invention, by particularly using a green LED as a light source, growth of algae other than algae containing fucoxanthin which cannot use green light, such as green algae, can be inhibited, thereby preventing contamination.

In the production method of the invention, the method of irradiation with light from a light source is not particularly limited, and examples of such methods include a method in which a light source is placed outside a culture vessel for culturing an alga containing fucoxanthin to irradiate the alga with light from the outside of the culture vessel, and a method in which a light source is placed inside a culture vessel for culturing an alga containing fucoxanthin to irradiate the alga with light from the inside of the culture vessel. The irradiation cycle with light is not particularly limited, and, for example, the light period may be 6 to 24 hours, preferably 12 to 24 hours. The light intensity of irradiation is not particularly limited, and unlike general culture conditions of algae, a higher intensity is preferred since the fucoxanthin content is more enhanced. More specifically, the irradiation is performed at a light intensity of 20 to 650 ppfd, more preferably at a light intensity of 300 to 600 ppfd.

In the production method of the invention, the culture of an alga is conducted with an apparatus that includes a culture vessel and a light source capable of emitting at least light corresponding to green. The culture vessel is not particularly limited, and examples of culture vessels include vessels formed of a resin, a glass, and a stainless steel. An alga may be put in such a culture vessel together with a culture solution, such as sea water or artificial sea water, and may be cultured at a water temperature suitable for the type of the alga, under stirring and aeration as needed. The culture period is not particularly limited, and may be 1 to 6 weeks and preferably 2 to 4 weeks. The culture place is not particularly limited, and is preferably an indoor place where light of the other wavelengths does not enter.

By culturing an alga in the above manner, a high fucoxanthin-containing alga can be obtained. Specifically, fucoxanthin in an alga is enriched to about 5 times or more, preferably to 10 to 30 times by the production method of the invention.

The thus obtained high fucoxanthin-containing alga maybe ate or drunk as it is if the alga can be ate or drunk, or the high fucoxanthin-containing alga may be used as a raw material of a food or drink like existing algae that can be ate or drunk. The form of the food or drink is not limited, and examples of forms include a powder, granules, tablets, soft capsules, hard capsules, and a beverage.

In addition, fucoxanthin may be extracted and/or purified from the high fucoxanthin-containing alga by a known method. The thus extracted fucoxanthin is applicable to various health foods, cosmetics, quasi-drugs, pharmaceuticals, and the like, and is usable for development or studies of such products.

As a preferred embodiment of the production method of the invention, conditions in the case where Okinawa mozuku or mozuku is used as an alga containing fucoxanthin are shown below.

Alga containing fucoxanthin: Okinawa mozuku or mozuku Light source capable of emitting at least light corresponding to green: green LED

Culture vessel: polycarbonate tank, acryl tank, raceway-type

Light intensity: 50 to 500 ppfd

Light cycle: 12 to 24 hours (light period)

Culture temperature: 20 to 30° C.

Culture period: 2 to 4 weeks

Seed rate: discoidal, 5 to 10 g/L

Aeration: 50 to 200 mL/min/L

The thus obtained Okinawa mozuku or mozuku contains fucoxanthin 10 times or more, preferably 20 to 100 times a content in a usual Okinawa mozuku or mozuku.

The high fucoxanthin-containing Okinawa mozuku or mozuku may be ate or drunk as it is, or may be processed into foods or drinks, such as mozuku in vinegar, salted mozuku, dried mozuku, boiled mozuku in soy (tsukudani), mozuku soup, mozuku seasoned powder (furikake), mozuku-containing noodle, mozuku Tempura, mozuku-containing boiled paste (chikiage), mozuku-containing rice, mozuku-containing rice cracker (senbei), and mozuku-containing ice cream. The fucoxanthin extracted and/or purified from the high fucoxanthin-containing Okinawa mozuku may be processed into a health food, such as a supplement, a cosmetic, a quasi-drug, or a pharmaceutical.

### EXAMPLES

The present invention will be described in detail below with reference to examples, but the present invention is not to be limited to the examples.

### Example 1

Culture of Okinawa Mozuku Using LEDs of Various Wavelengths as Light Source:

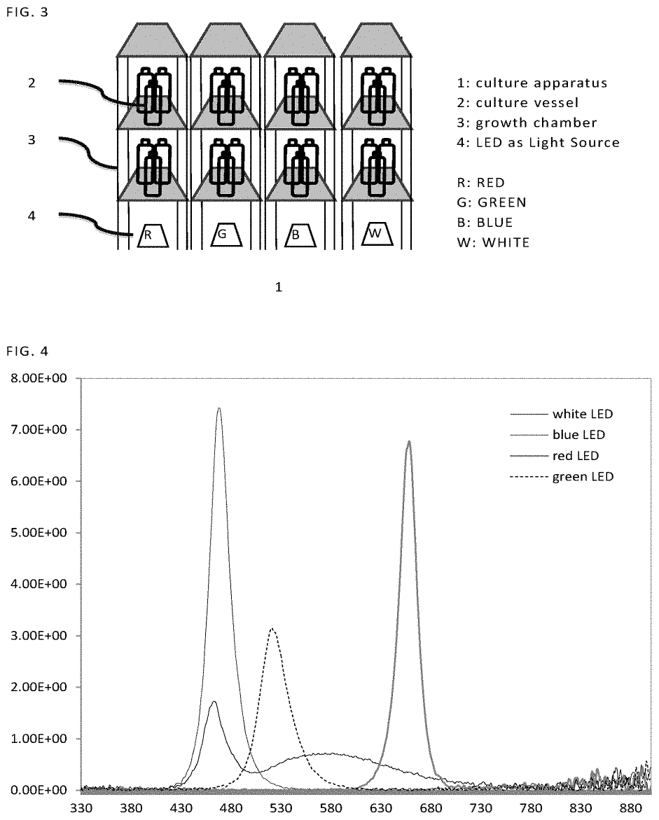

A sterilized sea water containing a culture solution was put in a 5-L transparent polycarbonate culture vessel, and 10 g of discoidal Okinawa mozuku (hereinafter abbreviated simply as “Okinawa mozuku”) was seeded therein, which was placed on an upper shelf or a lower shelf of a growth chamber having an LED on the bottom as shown in FIG. 3. FIG. 4 illustrates spectra of red, green, blue, and white LED panels (ISLM-150X150, manufactured by CCS Inc.) used in the culture. In the growth chamber, Okinawa mozuku was cultured at 25° C. for 4 weeks under aeration (500 mL/min) at a light cycle with a light period of 24 hours and a dark period of 0 hour (24L0D) . In the growth chamber, the light intensity was 30 ppfd on the upper shelf and 450 ppfd on the lower shelf. Note that the light intensity was measured at the center of the place where a culture vessel was to be placed in the state without the culture vessel (in the air). The light intensities were measured in the same manner also in the following Examples. Note that a light intensity in a culture vessel during an actual culture (in water) is known to be about 5 to 15% lower than a light intensity measured in the state without the culture vessel.

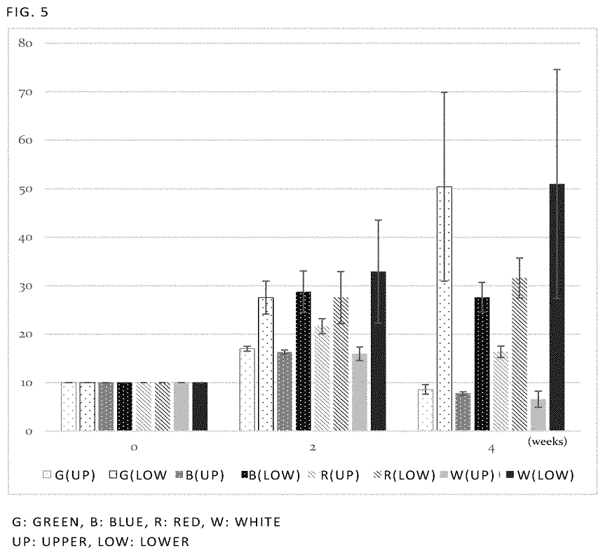

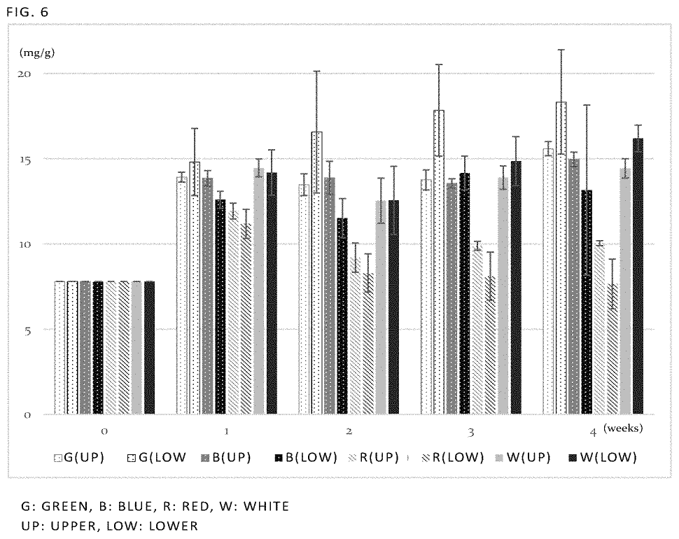

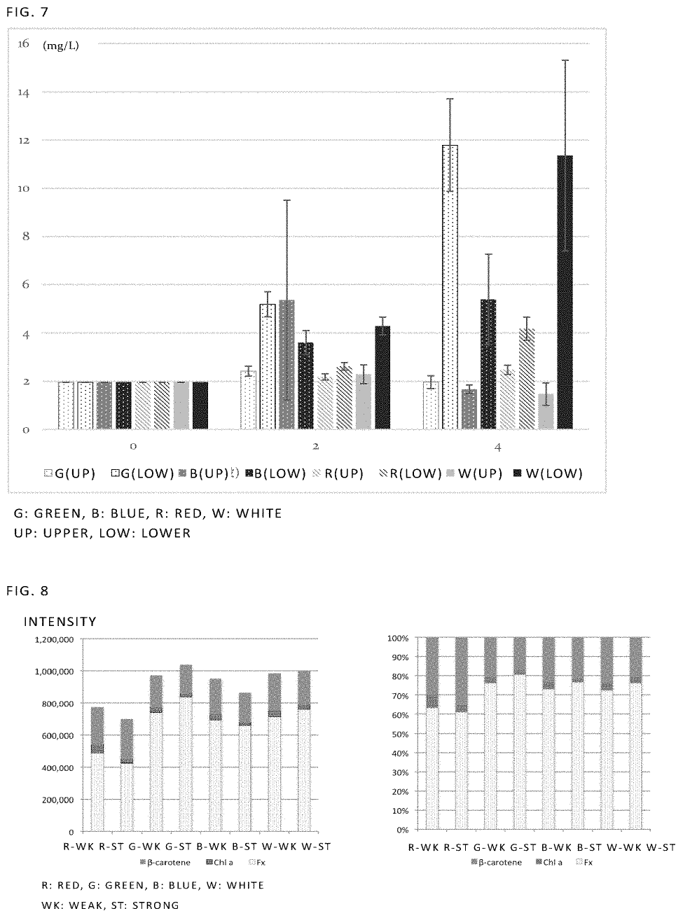

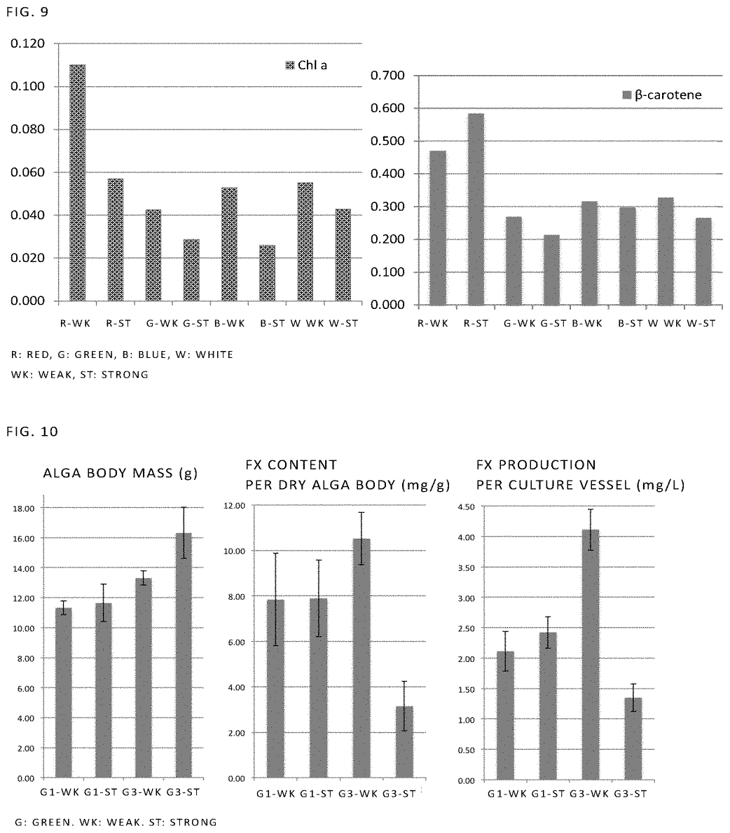

After culture, the alga body of Okinawa mozuku was collected with a plankton net and then the dry mass thereof was measured. The amount of fucoxanthin contained in the Okinawa mozuku was measured by HPLC with an ODS column after extracted with chloroform/methanol (1:1). The dry mass of the alga body after the extraction was measured to calculate the fucoxanthin content per alga body of the Okinawa mozuku. The pigment composition of fucoxanthin, chlorophyll a (Chl a), β-carotene, and violaxanthin (Vx) was also measured by HPLC with an ODS column. The productivity was indicated by a fucoxanthin yield per liter of the culture solution. The results are shown in FIGS. 5 to 9.

It was found that in the culture of Okinawa mozuku, the alga body production was higher at a larger quantity of light (higher light intensity) regardless of the wavelength of the light. The quantity of light is larger in an actual growth environment of mozuku than that from the light source in the growth chamber used in the test here. Accordingly, the productivity seemed to be further increased by culture with stronger light.

In culture of Okinawa mozuku, production of an alga body was affected by the light wavelength and green and white light provided a high alga body production. This matches the absorption wavelengths (400 to 550 nm) of a photosynthetic antenna protein (FCP) of mozuku, and thus the FCP seemed to have an influence on the alga body production. In contrast, red and blue which are in an absorption band of chlorophyll a/c did not largely affect the alga body production.

Similarly, the fucoxanthin content per alga body was affected by the light wavelength, and wavelengths corresponding to green and white led to a high fucoxanthin content per alga body. Since fucoxanthin is a pigment bound to FCP, the fucoxanthin content was higher with higher absorption and stronger light. It was found that a culture test should be conducted under a stronger light since the fucoxanthin content increases in proportion to the quantity of light.

In the study of an effect of light on the pigment composition, wavelengths corresponding to green and white led to compositions having a low ratio of chlorophyll a relative to fucoxanthin. This suggested that photosynthesis occurs around the FCP.

In this test, the fucoxanthin productivity per culture volume increased to about 10 times that of an existing culture with fluorescent light or natural light. In addition, it was found that the use of a green LED enables photosynthesis at only FCP and thus can be applied for prevention of contamination.

### Example 2

### Culture of Okinawa Mozuku Using White LED as Light Source:

Okinawa mozuku was cultured in the same manner as in Example 1 except for: using as a light source the white LED used in Example 1 or a special-order white LED (manufactured by CCS Inc., having a spectrum as illustrated in FIG. 2) in which light of wavelengths corresponding to green was strengthened in the white LED used in Example 1; and changing the culture period to 2 weeks.

The productivity of fucoxanthin was determined in the same manner as in Example 1. Then, the productivity of fucoxanthin in using the special-order white LED as a light source became 1.5 times that in using the general white LED as a light source.

### Example 3

### Culture of Mozuku Using Green LED as Light Source:

Sterilized sea water was put in a 5-L transparent polycarbonate culture vessel, and 10 g of discoidal mozuku (hereinafter abbreviated simply as “mozuku”) was seeded therein, which was placed on an upper shelf and a lower shelf of the same growth chamber as used in Example 1. One or three green LEDs were placed as a light source on the bottom of the growth chamber. In the case of one green LED, the light intensity was 16 ppfd (weak) on the upper shelf and was 240 ppfd (strong) on the lower shelf . In the case of three green LEDs, the light intensity was 250 ppfd (weak) on the upper shelf and was 6500 ppfd (strong) on the lower shelf. In the growth chamber, mozuku was cultured for 2 weeks at a light cycle with a light period of 24 hours and a dark period of 0 hour (24L0D) . During the culture, 1 ml of a nutrient salt TSP29 (1.25% sodium nitrate 1.25% sodium phosphate mixed solution) was added twice a week.

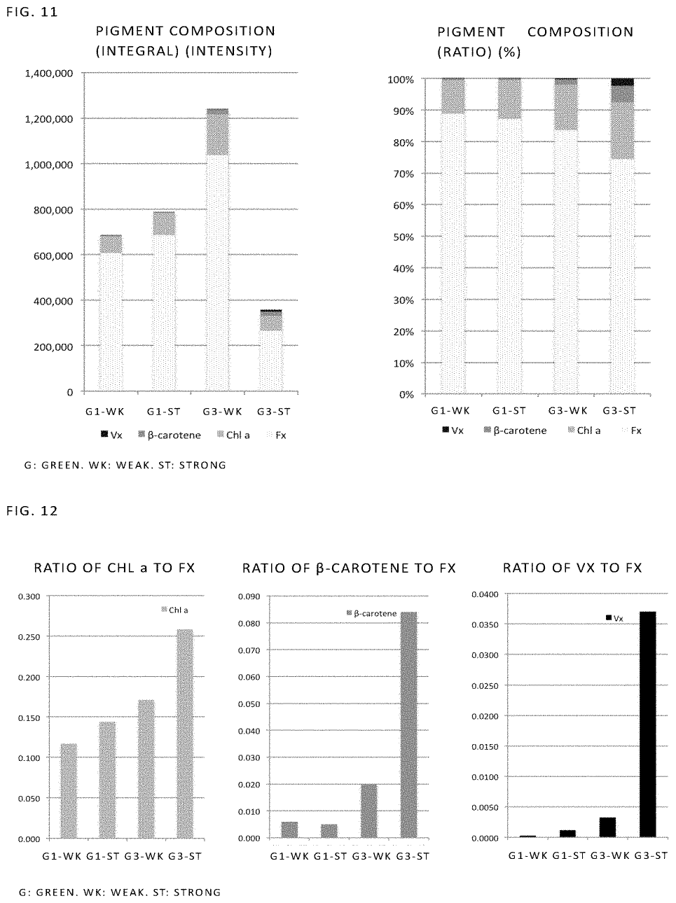

For the cultured mozuku, the mass of the alga body, the fucoxanthin content, and the fucoxanthin yield per liter of the culture solution were determined in the same manner as in Example 1. The results are shown in FIG. 10. The relationship between the light intensity and the pigment composition is shown in FIGS. 11 and 12.

It was found that the mass of alga body increased in proportion to the quantity of light through the culture conducted using one green LED or three green LEDs as a light source. The mass of alga body was large but the fucoxanthin contents per alga body and per culture vessel were low on the lower shelf with three green LEDs (650 ppfd) which was the case of the maximum quantity of light.

The production method of the invention can provide an alga having a high fucoxanthin content and thus is highly helpful for expanding the use of fucoxanthin which has various effects.

1. A method of producing a high fucoxanthin-containing alga, the method comprising culturing an alga containing fucoxanthin by irradiating the alga with light from a light source capable of emitting at least light corresponding to green.

2. The method of claim 1, wherein the light source capable of emitting at least light corresponding to green is a green LED.

3. The method of claim 1, wherein the irradiation is performed at a light intensity of 20 to 650 ppfd.

4. The method of claim 1, wherein the alga containing fucoxanthin is a brown alga or a diatom.

5. The method of claim 1, wherein the alga containing fucoxanthin is Okinawa mozuku (Cladosiphon okamuranus) or mozuku (Nemacystus decipiens).

6. A high fucoxanthin-containing alga produced by the method of claim 1.

7. A method of preventing contamination with an alga other than an alga containing fucoxanthin, the method comprising culturing an alga containing fucoxanthin by irradiating the alga with light from a light source capable of emitting at least light corresponding to green.

8. An apparatus for producing a high fucoxanthin-containing alga, the apparatus comprising a culture vessel and a light source capable of emitting at least light corresponding to green.

9. The method of claim 2, wherein the irradiation is performed at a light intensity of 20 to 650 ppfd.

10. The method of claim 2, wherein the alga containing fucoxanthin is a brown alga or a diatom.

11. The method of claim 2, wherein the alga containing fucoxanthin is Okinawa mozuku (Cladosiphon okamuranus) or mozuku (Nemacystus decipiens).

12. A high fucoxanthin-containing alga produced by the method of claim 2.
